(19) United States
(12) Patent Application Publication
(10) Pub. No.: US 2024/0277615 A1
(43) Pub. Date: Aug. 22, 2024

(54) SELF-EMULSIFYING COMPOSITION, PRODUCTION METHODS AND USES THEREOF

(71) Applicant: Universidade Da Beira Interior, Covilhã (PT)

(72) Inventors: Adriana OLIVEIRA DOS SANTOS, Covilhã (PT); Mariana CARVALHO FERNANDES, Alcaide (PT); Patrícia Sofia CABRAL PIRES, Covilhã (PT); Gilberto LOURENÇO ALVES, Castelo Branco (PT); Ana Carolina MARICOTO FAZENDEIRO, Covilhã (PT); Francisca MATOS SILVA PEREIRA NINA, Covilhã (PT); Maria De Fátima DA SILVA FERREIRA GOMES, Vila Nova De Famalicão (PT); Lina Isabel ESTEVES RODRIGUES, Covilhã (PT)

(73) Assignee: Universidade Da Beira Interior, Covilhã (PT)

(21) Appl. No.: 18/568,879

(22) PCT Filed: Jun. 9, 2022
(86) PCT No.: PCT/IB2022/055385

§ 371 Date: Dec. 11, 2023

(30) Foreign Application Priority Data
Jun. 11, 2021 (PT) 117283

(51) Int. Cl. A61K 9/107; A61K 8/06; A61K 8/37; A61K 8/41; A61K 31/22; A61K 31/366; A61K 31/40; A61K 31/405; A61K 31/4166; A61K 31/47; A61K 31/505; A61K 31/57; A61K 31/675; A61K 47/14; A61K 47/18; A61Q 19/00

(52) U.S. Cl. CPC A61K 9/107; A61K 8/06; A61K 8/375; A61K 8/416; A61K 31/22; A61K 31/366; A61K 31/40; A61K 31/405; A61K 31/4166; A61K 31/47; A61K 31/505; A61K 31/57; A61K 31/675; A61K 47/14; A61K 47/186; A61Q 19/00; A61K 2800/10

(57) ABSTRACT

The present disclosure relates to a self-emulsifying composition and/or a stable and homogeneous aqueous oil-in-water emulsion comprising a neutral hydrophilic surfactant and a combination of at least two hydrophobic excipients selected from the following list: glycerol monooleate, miglyol 812; propylene glycol monocaprylate; wherein 90% of the droplets comprise a size of less than 180 nm; and wherein the size of 50% of the droplets ranges from 90-120 nm. A pharmaceutical composition comprising said emulsion is also described.

### TECHNICAL FIELD

The present disclosure relates to a self-emulsifying composition and/or a stable and homogeneous nanometric oil-in-water emulsion of pharmaceutical and/or cosmetic interest. A pharmaceutical composition comprising said emulsion is also described.

### BACKGROUND

The emulsions with nanometric droplets can be of two types: microemulsions or nanoemulsions. Microemulsions are thermodynamically stable systems that form with certain proportions of lipids, surfactants, co-solvents and water, generally implying high amounts of surfactants and/or co-solvents. Microemulsions form spontaneously by simple mix of the components, but their droplets tend to disappear with increasing dilution in the continuous (external) phase. The high amount of surfactants and co-solvents reduces safety in internal use, especially in parenteral routes. Nanoemulsions are thermodynamically unstable preparations (unlike microemulsions) but with high kinetic stability compared to macroemulsions; aqueous nanoemulsions are usually composed of a lower content of hydrophilic surfactants and co-solvents than microemulsions, but usually require high energy methods for shearing and homogenizing the lipid droplets. Nanoemulsions can also be prepared by low-energy methods, but with some compromise in the type and amounts of surfactant and co-solvents (usually in high amounts) and in the degree of homogeneity, which is difficult to achieve in high degree.

In medicines, active substances with low aqueous solubility represent a bioavailability and formulation challenge. Simvastatin is an example of this type of active substance, being classified in the pharmacopeias as insoluble, and having a predicted water solubility of only 0.0122 mg/mL. Simvastatin belongs to the group of statins, a class of substances that inhibit Hydroxymethylglutaryl-coenzyme A reductase, usually taken orally in the form of tablets. However, simvastatin has low oral bioavailability and high plasma protein binding (94-98%). Additionally, simvastatin is extensively metabolized in the liver by two distinct pathways: hydrolysis of lactone to its active form, and oxidation by cytochrome P450 isoenzymes 3A4 and 2D6 [1]. In addition to the interest in the prevention and treatment of cardiovascular pathologies, there is strong preclinical evidence of the interest of simvastatin in the treatment of pathologies of the central nervous system (e.g. brain tumors and neurodegenerative diseases) and in the treatment of cancer of different etiologies. In general, all statins are well tolerated by patients, however some serious adverse effects associated with these drugs have been described. The most well documented serious adverse effect is muscle toxicity, which includes myopathy and rhabdomyolysis, and it may affect liver enzymes [2]. Although muscle toxicity is a relatively rare effect at the most usual therapeutic doses (40 mg/day) [3], at higher doses (80 mg/day) side effects are more frequent [2,4].

Some of the limitations of simvastatin and other drugs with low oral bioavailability, for example due to strong hepatic metabolism, could be overcome by resorting to administration by alternative routes to the oral route, including the nasal route (also called intranasal [i.n.] delivery). In fact, i.n. drug administration allows rapid systemic delivery of drugs and, partially, direct delivery from the nasal mucosa to the central nervous system (CNS) directly [5]. Therefore, this route of administration may allow the drugs to reach the CNS in therapeutic doses and in a non-invasive way. Furthermore, this route may allow to avoid the systemic side effects caused by the high doses of simvastatin that are necessary to administer to demonstrate a therapeutic effect on the brain.

As for previous compositions of simvastatin in the form of a nanoemulsion or for nasal administration, the following cases have been reported.

An oral simvastatin nanoemulsion at 18 mg/ml, with the following composition: lipophilic surfactant (Phospholipon 90) at 2% (m/V); 1.8% simvastatin (w/v); Capryol 90 as oil at 15% (V/V); Poloxamer 188 (Lutrol F68, hydrophilic surfactant) at 1% (w/v); and water enough for 100%. The preparation comprises dissolving simvastatin and Phospholipon 90 in Capryol 90, dissolving Poloxamer 188 in water, heating the oil and aqueous phases to 70° C., mixing the two phases in a high shear shaker (Ultra-Turrax at 9500 revolutions per minute, for 10 min) with subsequent ultrasonication (Ultrasonics, Sartorius, Mumbai, India). The simvastatin nanoemulsions in this publication were characterized by an average droplet size of 132 nm, with a PDI (Polydispersity Index) of 0.179 [6].

Lecithin and chitosan nanoparticles for nasal administration with approximately 1 mg of simvastatin/ml of preparation, which in the optimized composition consisted of: soy lecithin (Lipoid S45, 100 mg), simvastatin (50 mg), Maisine™ 35 oil-1 (Glyceryl Monolinoleate, 100 mg), Labrafac™ Lipophile WL 1349 Oil (Medium Chain Triglycerides, 100 mg), and 50 ml of a Chitosan Dispersion (Chitoclear FG, 95% DeacetylationDegree, Viscosity 45 cP) at 0.01% (m/V) in a 0.03N HCl solution. The preparation also involves the use of ethanol, which is however evaporated. The nanoparticles obtained were described with the following characteristics: average size of 204.5 nm, zeta potential of +48.45 mV, PDI of 0.098, and encapsulation efficiency of 98.52% [7].

Despite the existence of previous compositions in the form of a nanoemulsion or for nasal administration described in the prior art, these compositions were not able to surpass the limitation of low solubility of some active ingredients, and therefore comprises a lower concentration of such active ingredient. Additionally, the manufacturing processes described are complex, require high temperatures and the use of organic solvents and high shear equipment.

Contrary to all the expectations of one skilled in the art, it is possible to obtain a composition able to comprise a high dosage strength of an active ingredient in the final preparation, and such composition being homogeneous and stable, and obtained through a simple preparation process, with low temperatures and absence of organic solvents.

These facts are described in order to illustrate the technical problem solved by the embodiments of the present document.

### General Description

In an embodiment, the present disclosure relates to a composition that can be used as a liquid carrier of lipophilic or hydrophilic active substances in medicines, compatible with administration by various routes of administration. Additionally, the composition can be used as a fluid cosmetic preparation for cleansing and/or moisturizing the body, or as a vehicle for a wide range of active cosmetic ingredients, and incorporated in other bases. A preferred way of using it is the dissolution of drugs of low aqueous solubility, preferably drugs that are sufficiently soluble in a mixture of propylene glycol monocaprylate and glycerol monooleate; in order to increase their oral, transdermal, transmucosal (buccal, nasal, vaginal, rectal), and ophthalmic delivery, or to enable their solubilization in parenteral preparations (intravenous [i.v.], subcutaneous [s.c.], intramuscular, etc.), both inimmediate or r modified release preparations.

In an embodiment, the present disclosure relates to self-emulsifying compositions and their respective nanometric emulsions, of the nanoemulsion type, with a low content of (at least) a neutral hydrophilic surfactant and without co-solvents. These nanoemulsions can be prepared by simple mixture of the components, followed by refrigeration, resulting in a very homogeneous nanometric size (preferably less than 200 nm), with high physical stability (stability from the kinetic point of view).

A co-solvent is a water-miscible organic solvent, such as ethanol, glycerin, prolylene glycol, transcutol, polyethylene glycols, among others.

An active agent is a chemical agent capable of activity, preferably a pharmaceutical active agent or a cosmetic active agent.

Parenteral drug administration means any non-oral means of administration, comprising intramuscular, inhalation, i.n., intravenous, transdermal, submucosal, s.c., intraspinal, and intracapsular injections.

An aspect of the present disclosure relates to a self-emulsifying composition comprising
9-19% (w/w) of a neutral hydrophilic surfactant and;
81-91% (w/w) of a combination of at least two hydrophobic excipients in a mass ratio between 4:1 and 1.2:1,
propylene glycol monocaprylate as the first excipient;
a second excipient selected from the following list: glycerol monooleate, miglyol 812; glycerol monocaprylocaprate, soybean oil, type I glycerol monocaprylate, sorbitan monooleate, decyl oleate, glycerol monolinoleate, vitamin E, or mixtures thereof.

In an embodiment, the composition further comprises an active agent, preferably a pharmaceutical active agent or a cosmetic active agent.

In an embodiment, the active agent is lipophilic, hydrophilic, or mixtures thereof.

In an embodiment, the active agent is hydrophilic, and whose permeation of biological barriers such as mucous membranes and consequently absorption and bioavailability are improved by the formulation.

In an embodiment, the active agent is a statin, a steroid compound and/or an antiepileptic.

In an embodiment, the active agent is selected from a list comprising: atorvastatin, rosuvastatin, pravastatin, lovastatin, fluvastatin, pitavastatin, simvastatin, phenytoin, fosphenytoin, progesterone, nestorone (segesterone acetate) or mixtures thereof.

In an embodiment, the neutral hydrophilic surfactant is macrogolglycerol hydroxystearate (polyethylene glycol castor oil).

In an embodiment, the composition further comprises a cationic lipid; preferably wherein the cationic lipid is selected from a list consisting of cetalkonium chloride and dioleoyl-3-trimethylammonium propane.

In an embodiment, the concentration of the cationic lipid ranges from 0.01% to 1% (w/w); preferably 0.05 to 0.5% (w/w).

Another aspect of the present disclosure relates to an aqueous emulsion comprising said composition, wherein the concentration of the self-emulsifying composition is at least 0.1% (w/w).

In an embodiment, the concentration of the self-emulsifying composition is at least 1% (w/w), preferably 5% (w/w).

In an embodiment, the concentration of the self-emulsifying composition ranges from 4-50% (w/w), preferably 5-40% (w/w).

In an embodiment, 90% of the droplets of the aqueous emulsion comprise a dimension less than 200 nm, preferably less than 100 nm.

In an embodiment, 50% of the droplets of the aqueous emulsion comprise a dimension ranging from 90 to 120 nm.

In an embodiment, the aqueous emulsion comprises at least 50% (w/w) of water.

In an embodiment, the polydispersity index of the aqueous emulsion after cooling at 2-8° C. is less than or equal to 0.2; preferably less than 0.1.

In an embodiment, the aqueous emulsion further comprises a hydrophilic polymer.

In an embodiment, the concentration of the hydrophilic polymer ranges from 0.25% to 6% (w/w); preferably 2% (w/w).

In an embodiment, the hydrophilic polymer is selected from a list consisting of: albumin, polyvinylpyrrolidone, hydroxypropylmethylcellulose, polyethylene glycol, or mixtures thereof.

In an embodiment, the aqueous emulsion further comprises a salt; preferably the salt is sodium chloride.

In an embodiment, the concentration of the salt in the aqueous phase ranges from 0.1% to 0.9% (w/w); preferably 0.6% (w/w).

In an embodiment, the concentration of the active agent ranges up to 100 mg/g, preferably from 40-100 mg/g; more preferably from 60 to 100 mg/g.

In an embodiment, the concentration of the active agent ranges of the active agent ranges up to 10% (w/w), preferably from 4-10% (w/w), more preferably 6% to 10% (w/w).

In an embodiment, the concentration of the active agent ranges up to 100 mg/ml, preferably from 40-100 mg/ml; more preferably from 60 to 100 mg/ml.

In an embodiment, the aqueous emulsion comprises a co-solvent, an isotonic agent or a buffer solution, preferably malate buffer or phosphate buffer, or mixtures thereof.

In an embodiment, the emulsion remains homogeneous between 2 and 37° C.

In an embodiment, the aqueous emulsion, wherein the conditions being guaranteed that promote chemical and microbiological stability of the preparation; the emulsion maintains physical stability when stored at 2 to 25° C. for long periods of time; preferably periods longer than 6 months; more preferably for periods longer than 1 year.

Another aspect of the present disclosure relates to a pharmaceutical composition comprising the self-emulsifying composition and/or the aqueous emulsion herein described.

In an embodiment, the pharmaceutical composition is in atomized form.

In an embodiment, the pharmaceutical composition is either for enteral, parenteral or intranasal administration, preferably for intranasal administration.

Another aspect of the present disclosure relates to a cosmetic composition comprising the self-emulsifying composition and/or the aqueous emulsion herein described.

Another aspect of the present disclosure relates to a pharmaceutical vehicle comprising the self-emulsifying composition and/or the aqueous emulsion herein described.

In an embodiment, the nanoemulsion herein described is expected to be compatible with several administration routes in alternative to the oral route, including the nasal one.

In an embodiment, the present disclosure relates to a self-emulsifying composition capable of producing a stable and homogeneous aqueous oil-in-water emulsion, comprising a neutral hydrophilic surfactant and a combination of at least two hydrophobic excipients, selected from the following list: glycerol monooleate, miglyol 812; propylene glycol monocaprylate; wherein 90% of the droplets comprise a size of less than 180 nm; and wherein the size of 50% of the droplets ranges from 90-120 nm. A pharmaceutical composition comprising said self emulsifying composition or emulsion is also described.

In an embodiment, the present disclosure comprises a composition of pharmaceutical and/or cosmetic interest, able to form a nanoemulsion by a low energy method.

In one embodiment, the composition comprises an external aqueous phase and an internal phase, wherein the internal oily phase comprises dispersed droplets of diameter less than or equal to about 200 nm, preferably 100 nm, which comprises a mixture of propylene glycol monocaprylate (e.g., Capryol™ 90 from Gatefossé, Capmul PG-8 from Abitec) and another hydrophobic excipient from the glycerol monoster class, preferably glycerol monooleate (e.g., Imwitor® 948 from IOI Oleochemical, Peceol from Gatefossé, Capmul GMO-50 from Abitec) or glycerol monocaprylocaprate (e.g., Capmul MCM series from Abitec); preferably in a mass ratio between 4:1 and 1.2:1, respectively, and stabilized by a neutral hydrophilic surfactant in low proportion, preferably a polyethylene glycol castor oil, also known as Macrogolglycerol hydroxystearate (e.g., Kolliphor RH 40 or Kolliphor EL from BASF) in a ratio weight of about 1:5 or less, relative to the mixture of hydrophobic excipients, not requiring co-solvents. The formation of the nanoemulsion takes place spontaneously, for example in the addition of water, added to the mixture of hydrophobic surfactants and hydrophilic surfactant in a proportion greater than 35% (preferably 50%), divided in two additions, with slight agitation, and the homogeneity increases with the refrigeration (in refrigerator, 2-8° C.), not requiring any high energy procedure.

The composition of the present disclosure surprisingly allows a higher concentration of incorporation of active ingredients, and comprises lower average droplet size and lower PDI, hence greater homogeneity than previous art. Additionally, the manufacturing process does not need higher temperatures nor high shear agitation of ultrasonication.

In an embodiment, the composition of the present disclosure comprises simvastatin, a molecule poorly soluble in water. However, it showed to be soluble in the nanoemulsion described in the present disclosure at a concentration of at least 52 mg/g (about 4000 times more than in water).

In an embodiment, the formulation/composition of the present disclosure features a much higher dosage strength of simvastatin in the final preparation (up to about 50-fold), the smallest average droplet size, and a much simpler preparation process, including no use of organic solvents.

In one embodiment, the formulation/composition of the present disclosure contains only water as an aqueous phase and can be prepared concentrated, preferentially at about 50% (w/w) in water, being diluted after refrigeration. After significant dilution (at least about 10-fold), the homogeneity of the preparation is maintained even if refrigeration is removed.

In one embodiment, the fluidity of the nanoemulsion is high and can be adjusted, preferably with the addition of viscosifying agents, the pH and osmolality are adjustable by adding a suitable buffer and/or isotonizing system.

In an embodiment, it is possible the incorporation of any another type of water-soluble substance in the external phase, including a water-soluble drug.

In another embodiment, a hydrophilic polymer can be added (alone or in combination), such as (but not exclusively) serum albumin, polyethylene glycol 4000, polyvinylpyrrolidone or hydroxypropylmethylcellulose, for a final polymer concentration of 0.5 to 4% (w/w), preferably 2% (w/w), making refrigeration unnecessary to increase homogeneity, and allowing the incorporation of lipophilic active substances in high concentration (for example 5% (w/w) of simvastatin or 7.5% (w/w) of cholesterol, in emulsions with about 50% (w/w) of aqueous phase), maintaining spontaneous formation of an emulsion of homogeneous to highly homogeneous nanometer size. The use of a hydrophilic polymer may be further preferred for greater compatibility with a specific route of administration (e.g. parenteral, including nasal), mucoadhesion, active targeting, or modification of drug release.

The self-emulsifying composition/formulation and nanoemulsions described in the present disclosure are compatible with the oral and cutaneous routes given the current regulatory status of some of the excipients, at doses already established as safe, but potentially applicable to any route of administration of drugs (enteral or parenteral) after evaluation of their safety, and allows administration either by instillation or in the form of a spray, or included in other semi-solid or solid pharmaceutical preparations. Parenteral routes of administration are understood not only as injectable routes (intravenous.v., s.c., intramuscular, etc.), but all other routes in addition to enteric routes (cutaneous, transdermal, and other mucous membranes such as nasal, ophthalmic, vaginal, pulmonary, etc.).

In one embodiment, the preparation can be prepared, concentrated, and diluted after refrigeration. After significant dilution, the high homogeneity of the preparation is maintained even if refrigeration is removed. The droplet size and PDI obtained with refrigeration are maintained at room temperature if the refrigerated preparation is further diluted in room temperature water by at least 10-fold.

### BRIEF DESCRIPTION OF THE DRAWINGS

The following figures provide preferred embodiments for illustrating the disclosure and should not be seen as limiting the scope of invention.

FIG. 1: Schematic representation of the apparent viscosity of the preparations NE3A^{SIM} (without bovine serum albumin [BSA]) and NE3A^{SIM+BSA} (with BSA) as a function of rotation speed, at different temperatures. The temperatures evaluated were 4° C. (A), 20° C. (B) and 32° C. (C).

FIG. 2: Representation of drug release profile of NE3A^{PHT+BSA1%} (3.5 mg/g phenytoin) in in vitro horizontal Ussing chambers.

FIG. 3: Blood (A) and brain (B) concentration of phenytoin in mice administered in different ways and by different routes of administration. Data correspond to mean±standard deviation, N=2 to 5 mice at 3 collection points. The conditions of sample processing and validation of the analytical method, as well as the complete data of the fosphenytoin solution by i.v. and i.n. administration, shown for comparison, are published [8].

### DETAILED DESCRIPTION

The present disclosure relates to a self-emulsifying composition and/or a stable and homogeneous aqueous oil-in-water emulsion comprising a neutral hydrophilic surfactant and a combination of at least two hydrophobic excipients selected from the following list: glycerol monooleate, miglyol 812; propylene glycol monocaprylate; wherein 90% of the droplets comprise a size of less than 180 nm; and wherein the size of 50% of the droplets ranges from 90-120 nm. A pharmaceutical and/or cosmetic composition comprising said emulsion is also described.

The present description refers to a composition of pharmaceutical and/or cosmetic interest, in the form of a self-emulsifying composition and (or a stable, homogeneous to highly homogeneous, external phase aqueous nanoemulsion.

In the present disclosure, an emulsion is considered homogeneous when the PDI is equal to or less than 0.2, very homogeneous when this index is less than or equal to 0.1, and highly homogeneous if equal to or less than 0.05. According to the website of the company Malvern, which sells the Zetasizer equipment used to characterize droplet size, values below 0.05 are rarely obtained, except with highly homogeneous reference standards (https://www.malvernpanalytical.com/br/learn/knowledge-center/whitepapers/WP111214DLSTermsDefined). Greater uniformity in droplet size promotes more uniform absorption and less variability in the pharmacokinetics of the drug or active agent to be incorporated, as well as greater physical stability (reduced tendency for Ostwald ripening).

In one embodiment, the preparation of different emulsions is presented in series. The examples of achievement of the present disclosure correspond to compositions indicated from series 3 onwards. Preparation started by weighing all components of the preconcentrated mixture (hydrophobic excipients, hydrophilic surfactant and, if present, co-solvent) on a precision scale. When the active substance was added, it was added to the nanoemulsion preconcentrate (mixture of lipids and surfactant). In a next step, the required mass of aqueous phase (0.2 μm membrane filtered) was added in two steps. The last step in the preparation of the initial series emulsions was the extrusion of the formulation with a mini-extruder (Avanti Polar Lipids Mini Extruder®), passing it through a 200 nm pore polycarbonate membrane (19 mm, Whatman® Nuclepore™ Track-Etched, Sigma-Aldrich, Steinheim, Germany). This emulsion homogenization technique is known as “premix membrane emulsification” [9]. The extruder was maintained at a temperature of approximately 45° C. and 21 extrusion cycles were performed.

In one embodiment, the hydrodynamic mean diameter (Z-ave) and PDI were measured by dynamic light scattering, by cumulant analysis, with the Zetasizer NanoZS equipment from Malvern® (Malvern, UK) using the Zetasizer software (version 7.12). For the measurement, the samples were diluted (to about 0.1% of preconcentrate) with filtered ultrapure water (with a 0.2 μm filter) in duplicate, using disposable cuvettes. From each cuvette, three measurements were automatically performed. The measurement of the zeta potential was performed on the same equipment, diluting the samples in the same way. Measurements were performed at a temperature of 20° C. or 25° C. The viscosity and refractive index considered for the continuous phase were those of water.

The viscosity may be measured by different methods known in the art. In one embodiment of the present disclosure, viscosity was measured at various speeds (1 to 180 rpm) and at different temperatures (4° C., 20° C. and 32° C.) with the Brookfield DV3TRVCP® rheometer of cone-plate geometry, using the CPA-40Z and software version 1.1.13. Temperature control was ensured through the use of a thermostated bath. First, a volume of 500 μl of sample was measured into the dish, and then the temperature was allowed to stabilize. Finally, the viscosity of the samples was measured at predefined speeds.

In one embodiment, osmolality was measured in triplicate using the Osmomat 3000@ microosmometer from Gonotec GmbH (Berlin, Germany), previously calibrated with water and standards of 300 mOsmol/Kg and 850 mOsmol/Kg.

In one embodiment, the preparation of nanoemulsions using exclusively glycerol monooleate as lipid (series 1) required additional homogenization by a high-energy technique, in this case “premix membrane emulsification”, and ratios of hydrophilic surfactant:hydrophobic excipient:co-solvent greater than 1:1.75:0.25. With no co-solvent (ethylene glycol monoethyl ether, brand name Transcutol®) and a lower hydrophilic surfactant: hydrophobic excipient ratio (1:4) (NE1E formulation, Table 1), the formulation reversed to water-in-oil after one extrusion cycle (with no possibility to further dilute it in water) and had a semi-solid consistency at room temperature. The best formulation in this series was obtained with a relatively high hydrophilic surfactant:hydrophobic excipients:co-solvent ratio of 1:1:0.5 (formulation NE1A, Table 1).

TABLE 1
Composition and droplet size characterization of series 1
nanoemulsions.

| | Composition % | | | | Size before | | Size after | |
|---|---|---|---|---|---|---|---|---|
| | (w/w) | | | | extrusion | | extrusion | |
| Formulation | Imwitor | Kolliphor | Transcutol | Water | Z- | | Z-Ave | |
| code | 948 (%) | RH 40 (%) | (%) | (%) | Ave (nm) | PDI | (nm) | PDI |
| NE1A | 10 | 10 | 5 | 75 | NQ | 0.570 | 106.5 | 0.202 |
| NE1B | 15 | 10 | 2.5 | 72.5 | NQ | 0.431 | 186.6 | 0.310 |
| NE1C | 17 | 10 | 3 | 70 | NQ | 0.463 | 264.9 | 0.360 |
| NE1D | 17.5 | 10 | 2.5 | 70 | NQ | 0.682 | 194.6 | 0.228 |
| NE1E | 20 | 5 | 0 | 75 | NQ | 0.559 | — | — |

Imwitor 948, glycerol monooleate; Kolliphor RH 40, Polyethylene Glycol 40 Castor Oil; PDI, polydispersity index (English acronym); NQ, no quality, due to high heterogeneity; Transcutol, ethylene glycol monoethyl ether; Z-Ave, mean hydrodynamic diameter (by cumulant analysis). N = 1 or 2.

In an embodiment, in a subsequent phase (series 2) the active substance simvastatin was added to the best formulation of series 1 (NE1A). In a first attempt, 30 mg of simvastatin were added to 1 g of vehicle (300 mg of oil phase and 700 mg of aqueous phase, NE2^{SIM3%}), however, precipitation of simvastatin occurred after addition of the aqueous phase, meaning that the substance was in too high a quantity. In a second attempt, 20 mg of simvastatin per g of vehicle (NE2^{SIM2%}) was added, with no precipitation occurring. This formulation was characterized for hydrodynamic droplet diameter and PDI (Table 2).

TABLE 2
Composition and droplet size characterization of series 2
nanoemulsions.

| | Composition | | | | | Size before | | Size after | |
|---|---|---|---|---|---|---|---|---|---|
| | | Kolliphor | | | Amount of | extrusion | | extrusion | |
| Form. | Imwitor | RH 40 | Transcutol | Water | drug per g of | Z-Ave | | Z-Ave | |
| code | 948 (%) | (%) | (%) | (%) | vehicle (mg) | (nm) | PDI | (nm) | PDI |
| NE2^{SIN2%} | 17.5 | 10 | 2.5 | 70 | 20 | NQ | 0.618 | 231 | 0.278 |

| | | |
|---|---|---|
| NE2^{SIN3%} | 30 | NA |

Imwitor 948, glycerol monooleate; Kolliphor RH 40, Polyethylene Glycol 40 Castor Oil; NA, Not applicable; PDI, polydispersity index; NQ, No quality, due to high heterogeneity; Transcutol, ethylene glycol monoethyl ether; Z-Ave, mean hydrodynamic diameter (by cumulant analysis). N = 2 replicates.

In one embodiment, to increase the amount of dissolved drug, a new hydrophobic excipient (propylene glycol monocaprylate) was tested in addition to or in place of the previous one and the proportions of the excipients were varied (series 3). The results of characterization of the droplet size dispersion in the formulations of this series are presented in Table 3. Note that the formulations with the mixture of the two hydrophobic excipients (NE3A and NE3B) or with more Kolliphor RH 40 (NE3C to NE3E) already presented nanometric size before extrusion, which only influenced the PDI. The NE3A formulation demonstrated an already reduced PDI before extrusion (close to 0.2) and almost 0.1 after extrusion. In addition, it had a low ratio of hydrophilic surfactant: hydrophobic excipients (1:5) and no co-solvent.

TABLE 3
Composition and droplet size characterization of series 3
formulations (room temperature).

| | | | | | | Size before | | | |
|---|---|---|---|---|---|---|---|---|---|
| | Composition | | | | | extrusion | | Size after extrusion | |
| Form. | Capryol | Imwitor | Kolliphor | Transc. | Water | Z-Ave | | Z-Ave | |
| code | 90 (%) | 948 (%) | RH 40 (%) | (%) | (%) | (nm) | PDI | (nm) | PDI |
| NE3A | 15 | 10 | 5 | 0 | 70 | 152.8 | 0.214 | 172.9 | 0.114 |
| NE3B | 20 | 5 | 5 | 0 | 70 | 187.2 | 0.360 | 183.8 | 0.254 |
| NE3C | 17.5 | 0 | 10 | 2.5 | 70 | 80.6 | 0.434 | 102.3 | 0.276 |
| NE3D | 20 | 0 | 7.5 | 2.5 | 70 | 148.2 | 0.412 | 98.2 | 0.272 |
| NE3E | 22.5 | 0 | 7.5 | 0 | 70 | 120.4 | 0.674 | 95.3 | 0.383* |
| NE3F | 25 | 0 | 5 | 0 | 70 | NQ | 0.646 | 130.0 | 0.214** |
| NE3G | 27 | 0 | 3 | 0 | 70 | NQ | 0.673 | 196.6 | 0.337* |

Capryol 90, propylene glycol monocaprylate; Formulation, Formulation; Imwitor 948, glycerol monooleate; Kolliphor RH 40, Polyethylene Glycol 40 Castor Oil; PDI, polydispersity index; NQ, No quality, due to high heterogeneity; Trans., Transcutol, ethylene glycol monoethyl ether; Z-Ave, mean hydrodynamic diameter (by cumulant analysis).
*p < 0.05 and
**p < 0.01, ANOVA with Sidak multi-comparative test.

In one embodiment, in the formulations of series 4, the preconcentrate composition was identical to the NE3A formulation, and only the proportion of aqueous phase was varied (Table 4), with the aim of developing a formulation with a higher percentage of preconcentrate, in order to dissolve as much of the active substance as possible. Furthermore, in this series, the aqueous phase was further modified, using 70 mM malate buffer pH 5 (to adjust both pH and osmolality to the area of greatest drug stability and to the nasal administration route). The percentage of aqueous phase was varied between 40% and 70% and, for each formulation, the hydrodynamic diameter, PDI and osmolality were evaluated (Table 4). Additionally, some formulations showed greater transparency after being placed in the refrigerator (about 4° C.), which is indicative of a smaller hydrodynamic diameter. To verify the effect of temperature on size, dilution for size measurement was made from extruded and non-extruded NE3A-50% nanoemulsion at different temperatures. The refrigerated nanoemulsions showed a significantly lower hydrodynamic diameter and PDI than the formulations that were at room temperature at the time of dilution (analysis of variance with Sidak's multicomparative test, Table 4). The best condition was even without extrusion, where the nanoemulsions having an average droplet size of less than 100 nm were in fact very homogeneous (PDI less than 0.1), or even highly homogeneous (PDI less than 0.05).

TABLE 4
Composition and characterization of droplet size and osmolality of
the series 4 formulations, with variation of the proportion of preconcentrate to the
aqueous phase.

| | | Without extrusion | | | | With extrusion | | | | |
|---|---|---|---|---|---|---|---|---|---|---|
| | Aqueous | 4° C. | | RT | | 4° C. | | RT | | Osmolal. |
| Form. | phase | Z-Ave | | Z-Ave | | Z-Ave | | Z-Ave | | (mOsmol/ |
| code | (%, w/w) | (nm) | PDI | (nm) | PDI | (nm) | PDI | (nm) | PDI | Kg) |
| NE3A^{40%} | 40 | 116.3 | 0.146 | — | — | 104.6 | 0.062 | — | — | 3236 |
| NE3A^{50%} | 50 | 89.4 | 0.040 | 139.4 | 0.246 | 101.3 | 0.065 | 150.4 | 0.156 | 592 |
| NE3A^{55%} | 55 | 88.3 | 0.044 | — | — | 88.0 | 0.042 | — | — | 485 |
| NE3A^{60%} | 60 | 75.4 | 0.073 | — | 60 | 557 | 0.040 | — | — | 397 |
| NE3A^{70%} | 70 | 70.1 | 0.096 | — | — | 71.2 | 0.045 | — | — | 302 |

Form., Formulation; Osmolal., Osmolality, mean of 5 measurements; PDI, polydispersity index; RT, room temperature, (close to 20° C.); Z-Ave, mean hydrodynamic diameter by cumulant analysis.

In an embodiment, NE3A50% (50% water) concentrated nanoemulsion can be diluted after cooling, in water at room temperature. After significant dilution (at least about 10-fold), the obtained nanoemulsion remains homogeneous, and is maintained even if refrigeration is removed (Table 5).

TABLE 5
Characterization of the formulations obtained by diluting the
refrigerated NE3A^{50%} formulation in water at room temperature.

| Dilution factor | Z-Ave ± SD (nm) | PDI ± SD | N |
|---|---|---|---|
| 1/2 | 127.8 ± 7.1 | 0.437 ± 0.037 | 4 |
| 1/10 | 96.5 ± 8.7 | 0.122 ± 0.014 | 4 |
| 1/100 | 93.8 ± 2.1 | 0.058 ± 0.008 | 4 |
| 1/500 | 92.2 ± 2.9 | 0.078 ± 0.020 | 4 |

In another embodiment, in series 5, the composition of the preconcentrate mixture was identical to that of the NE3A formulation, but it was tested the addition of a lipophilic drug and a different aqueous phase. After preparing the preconcentrated mixture (with or without drug), part of the aqueous phase (30 mM malate buffer, pH 5) was added in a proportion of 40% (w/w) of the desired total and stirred. Finally, water or an aqueous solution of albumin (10% w/w) was added, in the proportion of 10% (w/w) of the desired total, resulting in a final proportion of preconcentrated drug-free mixture to the aqueous phase of 1:1, and a final albumin concentration of 1% (w/w). In this series, extrusion homogenization was not performed.

In one embodiment, in the attempt to solubilize 60 mg of simvastatin per g of vehicle (500 mg of pre-concentrated mixture) precipitation of the drug occurred after addition of the aqueous phase, however this did not happen at the ratio of 55 mg of simvastatin to 1 g of vehicle, which represents a high dosage strength (52 mg/g of preparation), higher than similar formulations reported in the literature. A previously reported emulsion in the literature comprised a much lower strength of this drug (18 mg/ml nanoemulsion) [6]. The nanometer systems described in the literature for i.n. delivery of simvastatin also report much lower strength of 1 mg/mL [7,10] and 20 μg/mL in the internal phase [11].

In one embodiment, the effect of adding a drug in high concentration to the formula on the characteristics of the nanoemulsion of the present disclosure, by dissolution in the mixture of hydrophobic excipients and surfactant (preconcentrate), was tested in 3 independent batches. The addition of simvastatin to the albumin-free formulation caused a significant increase in the size and in the PDI of the nanoemulsion (p<0.01, ANOVA with Sidak's multicomparative test), but not in the albumin-containing formula (Table 6). Osmolality, on the other hand, was not influenced by the drug or albumin, with an average range of between 530 and 590 mOsmol/kg in all cases. The drug-free and albumin-free formulation showed a slightly negative zeta potential (considered neutral). Both simvastatin and albumin led to a decrease in zeta potential, but only slightly. The acidic pH of the malate buffer used is close to, but above, the isoelectric point of albumin, which will already have some net negative charge, but not much. However, when the drug and albumin are both present in the formulation there appears to be a steric hindrance effect caused by the albumin, thus decreasing the effect of simvastatin loading and, consequently, its zeta potential. In short, in this embodiment, the formulation composed of the optimized preconcentrate and albumin in the external phase can contain a high amount of drug, has an average hydrodynamic diameter close to 100 nm and is very homogeneous (PDI less than 0.1).

TABLE 6
Composition and droplet size characterization of the series 5
formulations. The formulation was refrigerated (at 4° C.) at the time of dilution for the
measurement of droplet size and zeta potential. Data with replicates correspond to
the mean ± standard deviation.

| | Composition (% w/w) | | | | | | | | | |
|---|---|---|---|---|---|---|---|---|---|---|
| | | | Malate | | Sol. | Size | | | Zeta | |
| Formulation | | | buffer | | BSA | Z-Ave | | | Potential | N |
| code | Preconc. | Sim. | pH 5 | Water | 10% | (nm) | PDI | N | (mV) | — |
| NE3A^{pH5} | 50 | 0 | 40 | 10 | — | 93.9 ± 6.3 | 0.039 ± 0.002 | 2 | −3.9 | 1 |
| NE3A^{BSA1%} | 50 | 0 | 40 | — | 10 | 98.1 ± 7.9 | 0.040 ± 0.002 | 2 | −9.0 | 1 |
| NE3A^{SIM} | 47.4 | 5.2 | 37.9 | 9.5 | — | 143.6 ± 9.0 | 0.450 ± 0.001 | 3 | −17.3 ± 4.9 | 3 |
| NE3A^{SIM+BSA1%} | 47.4 | 5.2 | 37.9 | — | 9.5 | 113.0 ± 7.9 | 0.073 ± 0.017 | 3 | −11.5 ± 1.1 | 3 |

BSA, Bovine serum albumin; PDI, polydispersity index; Preconc., pre-concentrate; Sim., simvastatin; Z-Ave, mean hydrodynamic diameter (by cumulant analysis).

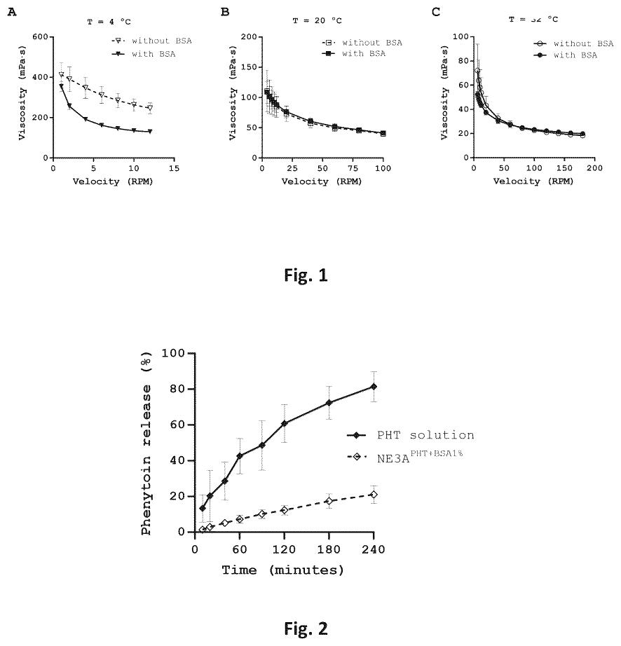

In one embodiment, the viscosity of the preparations was studied in the formulations of series 5, with and without albumin. In addition, the effect of temperature on viscosity was studied. In drug formulations (with and without albumin), the test was repeated on 3 independent formulations. The formulations showed non-Newtonian pseudoplastic-like behavior at all temperatures (FIG. 1).

Zero shear viscosity (at rest) was also estimated, calculating the ordinate at the origin from the linear regression function of the data in logarithmic form (Table 7). Estimated zero shear viscosity decreased significantly as temperature increased, as expected. However, albumin does not seem to have had a substantial influence on the values of zero shear viscosity (at temperatures of 4° C. and 32° C. it is not possible to compare the values estimated by the linear regressions because the slopes were significantly different).

TABLE 7
Zero shear viscosity estimated by linear regression
for temperatures of 4, 20 and 32° C. The mean
± 95% confidence interval (mPa · s) is shown.
R² > 0.98 for all lines, using the average of Y at each point.

| | 4° C. | 20° C. | 32° C. |
|---|---|---|---|
| With BSA | 345.1 | 177.4 | 87.9 |
| | (+20.1; −20.5) | (+24.4; −21.10) | (+5.6; −5.1) |
| Without BSA | 433.6 | 173.8 | 140.9 |
| | (+56.3; −49.8) | (+38.1; −30.9) | (+23.9; −20.1) |

BSA, bovine serum albumin. Brookfield DV3TRVCP ® rheometer of cone-plate geometry was used, using the CPA-40Z and software version 1.1.13. Temperature control was ensured through the use of a thermostated bath. The viscosity may be measured by different methods known in the art.

In another embodiment, the present disclosure may contain one of several hydrophilic polymers in the external phase, which promote the obtaining of homogeneous to highly homogeneous nanoemulsions, even in the absence of refrigeration (Table 8).

TABLE 8
Composition and droplet size characterization of variants of formula
NE3A with different hydrophilic polymers in the aqueous phase. Preconcentrate (50%)
formed by propylene glycol monocaprylate: glycerol monooleate: Kolliphor RH 40 in a 3:2:1 mass ratio.

| | External phase composition (50%) | | | | | RT | | 4° C. | |
|---|---|---|---|---|---|---|---|---|---|
| Formulation | BSA | | | | Carbopol | Z-Ave | | Z-Ave | |
| code | pH 7 | HPMC | PVP | PEG | 971P | (nm) | PDI | (nm) | PDI |
| NE3A^{BSA 2%} | 4% | — | — | — | — | 98 | 0.044 | 102 | 0.050 |
| NE3A^{HPMC 1%} | — | 2% | — | — | — | 98 | 0.092 | 99 | 0.083 |
| NE3A^{HPMC 0.25%} | — | 0.5% | — | — | — | 91 | 0.047 | 97 | 0.125 |
| NE3A^{PVP 1%} | — | — | 2% | — | — | 86 | 0.037 | 90 | 0.032 |
| NE3A^{PEG 1%} | — | — | — | 2% | — | 103 | 0.080 | — | — |
| NE3A^{PEG 2%} | — | — | — | 4% | — | 92 | 0.059 | 111 | 0.096 |
| NE3A^{PEG 3%} | — | — | — | 6% | — | 106 | 0.076 | — | — |
| NE3A^{Carbopol 0.1%} | — | — | — | — | 0.2% | 163 | 0.182 | 113 | 0.243 |
| NE3A^{BSA 2% PVP 2%} | 2% | — | 2% | — | — | 102.7 | 0.079 | 84.73 | 0.07 |
| NE3A^{BSA 2% PEG 4%} | 2% | — | — | 4% | — | 144.4 | 0.298 | 95.36 | 0.145 |
| NE3A^{PVP 2% PEG 4%} | — | — | 2% | 4% | — | 113.8 | 0.183 | 104.4 | 0.216 |

BSA, Bovine serum albumin; HPMC, hydroxypropylmethylcellulose; PDI, polydispersity index; PEG, polyethylene glycol 4000; PVP, polyvinylpyrrolidone; RT, room temperature, (close to 20° C.); Z-Ave, mean hydrodynamic diameter by cumulant analysis.

In another embodiment, the ratio of hydrophobic excipients:Kolliphor RH 40 can be varied (series 6) within a specific range without loss of nanometer size and low PDI (Table 9). The nanometer size (about 200 nm or less) and low PDI value were possible at different temperatures up to about the hydrophobic excipients:Kolliphor RH 40 ratio of 10:1, which corresponds to a concentration of Kolliphor RH 40 in the prepared emulsion with 50% aqueous phase of only 4.5%. The optimal zone of average droplet size around 100 nm and PDI less than 0.1 regardless of temperature was obtained with hydrophobic excipients/Kolliphor RH 40 ratios between roughly 4.17 and 10.

TABLE 9
Composition and characterization of the series 6 droplet size,
with NE3 formula variants by varying the hydrophobic
excipients/Kolliphor RH 40 ratio in the preconcentrate (50%).
The propylene glycol monocaprylate/glycerol monooleate
ratio was kept constant at 1.5. As the external phase a
4% (50%) PEG 4000 aqueous solution was used.

| | Ratio | | | | | | |
|---|---|---|---|---|---|---|---|
| | hydro- | | | | | | |
| | phobic | | | | | | |
| | excipients/ | | | | | | |
| For- | Kolliphor | RT | | 4° C. | | 37° C. | |
| mulation | RH | Z-Ave | | Z-Ave | | Z-Ave | |
| code | 40 | (nm) | PDI | (nm) | PDI | (nm) | PDI |
| NE3H^{PEG 2%} | 16.56 | 366 | 0.174 | NQ | 0.333 | 523 | 0.343 |
| NE31 | 12.51 | 300 | 0.312 | 270 | 0.306 | 0.586 | 0.586 |
| PEG^{2%} | | | | | | | |
| PEG^{2%} | 10.00 | 181 | 0.071 | 175 | 0.078 | 209 | 0.080 |
| NE3K^{PEG 2%} | 7.13 | 182 | 0.143 | 146 | 0.054 | 182 | 0.072 |
| NE3L^{PEG 2%} | 5.56 | 111 | 0.076 | 116 | 0.051 | 121 | 0.078 |
| NE3A | 5.00 | 102 | 0.075 | 107 | 0.056 | 103 | 0.084 |
| PEG^{2%} | | | | | | | |
| NE3M^{PEG 2%} | 4.56 | 102 | 0.079 | 99 | 0.079 | 99 | 0.078 |
| NE3N^{PEG 2%} | 4.17 | 100 | 0.114 | 95 | 0.082 | 94 | 0.098 |
| NE3O^{PEG 2%} | 3.33 | 116 | 0.180 | 116 | 0.143 | 114 | 0.170 |

NQ, No Quality; PDI, polydispersity index; PEG, polyethylene glycol 4000; RT, room temperature, (close to 20° C.); Z-Ave, mean hydrodynamic diameter (by cumulant analysis).

In another embodiment, replacing Kolliphor RH 40 with other surfactants (Kolliphor EL, Kolliphor HS 15, Labrasol ALF, Tween 20, Tween 60, Tween 80, Kolliphor P124, Tyloxapol), using an identical proportion of excipients as formula NE3A, resulted in much more heterogeneous emulsions (PDI>0.4) and with an average diameter greater than 200 nm. However, by optimizing the ratio of excipients, for example by optimizing the ratio of propylene glycol monocaprylate:glycerol monooleate to 1.86:1, it was possible to achieve homogeneous to highly homogeneous nanometer emulsions, as demonstrated with the Kolliphor EL in the series 7, in which the ratio of propylene glycol monocaprylate:glycerol monooleate was kept constant and the ratio of hydrophobic excipients: Kolliphor EL varied as well as the composition of the external phase (Table 10). The optimized formula in this series corresponds to NE4D^{PEG 2%}, with a hydrophobic excipients/Kolliphor EL mass ratio of 6.2. In this series, several factors besides composition were important, such as temperature (smaller average size and PDI at 4° C.) and consecutive dilution of the emulsion with water (smaller average size and PDI with increasing dilution, becoming highly homogeneous).

TABLE 10
Composition and droplet size characterization of series 7, with
replacement of Kolliphor RH 40 by Kolliphor EL in the preconcentrate (50% or 25%).
The propylene glycol monocaprylate/glycerol monooleate ratio was kept constant at
1.86. An aqueous solution of 4% PEG 4000 or 4% PEG4000 and water was used as the
external phase.

| | Composition (%) | | | | | | RT | | 4° C. | |
|---|---|---|---|---|---|---|---|---|---|---|
| Formulation | | Imwitor | | | | | Z-Ave | | Z-Ave | |
| code | Capryol 90 | 948 | Kolliphor EL | PEG 2% | PEG 4% | Water | (nm) | PDI | (nm) | PDI |
| NE4A^{PEG 1%} | 29.55 | 15.9 | 4.55 | 50 | — | — | NQ | 0.909 | 218 | 0.187 |
| NE4B ^{PEG 1%} | 29 | 15.65 | 5.35 | 50 | — | — | 274 | 0.592 | 188 | 0.096 |
| NE4C ^{PEG 1%} | 28.5 | 15.35 | 6.15 | 50 | — | — | 176 | 0.533 | 179 | 0.061 |
| NE4D ^{PEG 1%} | 28 | 15.1 | 6.9 | 50 | — | — | 159 | 0.140 | 164 | 0.058 |
| NE4A ^{PEG 2%} | 29.55 | 15.9 | 4.55 | — | 50 | — | 262 | 0.674 | 199 | 0.101 |
| NE4B ^{PEG 2%} | 29 | 15.65 | 5.35 | — | 50 | — | 209 | 0.204 | 201 | 0.077 |
| NE4C ^{PEG 2%} | 28.5 | 15.35 | 6.15 | — | 50 | — | 176 | 0.073 | 187 | 0.075 |
| NE4D ^{PEG 2%} | 28 | 15.1 | 6.9 | — | 50 | — | 167 | 0.087 | 167 | 0.064 |
| NE4E ^{PEG 2%} | 27.55 | 14.85 | 7.65 | — | 50 | — | 173 | 0.144 | 166 | 0.108 |
| NE4F ^{PEG 2%} | 27.1 | 14.6 | 8.35 | — | 50 | — | 211 | 0.297 | 217 | 0.300 |
| NE4A^{PEG 2% dil.} | 14.77 | 7.95 | 2.275 | — | 25 | 50 | — | — | 156 | 0.043 |
| NE4B^{PEG 2% dil.} | 14.5 | 7.825 | 2.675 | — | 25 | 50 | — | — | 145 | 0.038 |
| NE4C^{PEG 2% dil} | 14.25 | 7.675 | 3.075 | — | 25 | 50 | — | — | 138 | 0.051 |
| NE4D^{PEG 2% dil.} | 14 | 7.55 | 3.45 | — | 25 | 50 | — | — | 130 | 0.027 |

Capryol 90, propylene glycol monocaprylate; Imwitor 948, glycerol monooleate; NQ, No quality, due to high heterogeneity; PDI, polydispersity index; PEG, polyethylene glycol 4000; RT, room temperature (close to 20° C.); Z-Ave, mean hydrodynamic diameter by cumulant analysis).

In yet another embodiment, the minor hydrophobic excipient in NE3A^{PEG 2%} was replaced by others, maintaining the mass proportions between excipients, some of which resulted in homogeneous or very homogeneous nanoemulsions (Table 11); and others, however resulting in heterogeneous emulsions of propylene glycol dicaprilocaprate, bis-diglyceryl 2-polyacyladipate, isopropyl myristate, castor oil).

TABLE 11
Composition and droplet size characterization of series
8, with substitution of glycerol monooleate (preconcentrate
^{PEG 2%}). The proportion of excipients in the preconcentrate
was kept constant. As the external phase (50%) a 4%
aqueous PEG 4000 solution was used.

| | RT | | 4° C. | |
|---|---|---|---|---|
| Excipient used instead | Z-Ave | | Z-Ave | |
| of Imwitor 948 | (nm) | PDI | (nm) | PDI |
| Glycerol monocaprylocaprate | 84.6 | 0.070 | 84.8 | 0.064 |
| (Capmul MCM) | | | | |
| Soybean oil | 148.4 | 0.128 | 145.6 | 0.113 |
| Glycerol Monocaprylate, Type I | 121.0 | 0.167 | 134.0 | 0.121 |
| (Imwitor 988) | | | | |
| Sorbitan Monooleate (Span 80) | 88.4 | 0.167 | 89.0 | 0.141 |
| Decyl Oleate (Cetiol V) | 140.6 | 0.196 | 140.0 | 0.171 |
| Glycerol Monolinoleate | 198.3 | 0.145 | 145.6 | 0.236 |
| (Maisine ® CC) | | | | |
| Vitamin E | 147.2 | 0.203 | 144.9 | 0.194 |

PDI, polydispersity index; PEG, polyethylene glycol 4000; RT, room temperature, (close to 20° C.); Z-Ave, mean hydrodynamic diameter by cumulant analysis).

An example of the utility of the present disclosure is the delivery of simvastatin by different routes of administration, which can be applied even to situations of unavailability of the oral route, or possible special interest in the delivery of simvastatin to the brain via the nasal route. The increase in the central bioavailability of the drug could allow the treatment of brain tumors and neurodegenerative diseases, in less than 4 administrations of 0.2 ml in each nostril per day, the number necessary to obtain the daily therapeutic dose of 80 mg with the NE3A^{SIM+BSA1%}.

Other useful examples of the present disclosure are the delivery of other active substances of low aqueous solubility (in addition to simvastatin), such as phenytoin, or to represent the class of steroids or other molecules with very high log P, cholesterol (Log P=7) and Nestorone (segesterone acetate) (Table 12). It should be noted that, surprisingly, it was possible to obtain a cholesterol dosage strength of 7.5% (w/w), without loss of the nanometric character and high homogeneity (low PDI) in the nanoemulsions. Hydrophilic drugs such as fosphenytoin can also be transported in the external phase, with the nanoemulsion functioning as a permeation promoter (Table 12).

TABLE 12
Composition and droplet size characterization of nanoemulsions containing examples of dissolved drugs or chemical compounds.
Preconcentrate (50%) formed by propylene glycol monocaprylate:glycerol monooleate:Kolliphor RH 40 in a
3:2:1 mass ratio and compound dissolved in a final percentage concentration in the emulsion equal to the value indicated
in the table. As external phase (50%) an aqueous solution of albumin at 2% or 4%, pH 7 (20 mM phosphate buffer) was used.

| | Composition | | RT | | 4° C. | |
|---|---|---|---|---|---|---|
| | Dissolved compound | | Z-Ave | | Z-Ave | |
| Formulation code | name | % (w/w) | (nm) | PDI | (nm) | PDI |
| NE3A^{CHOL5%+BSA1%} | Cholesterol | 5 | 110 | 0.064 | — | — |
| NE3A^{CHOL7.5%+BSA1%} | Cholesterol | 7.5 | 138 | 0.097 | 111 | 0.075 |
| NE3A^{PHT+BSA1%} | Phenytoin | 0.35 | 94 | 0.042 | 90 | 0.029 |
| NE3A^{PHT+BSA2%} | Phenytoin | 0.35 | 124 | 0.144 | 89 | 0.021 |
| NE3A^{FOS+BSA2%} | Fos- | 4.25 | 85 | 0.177 | 61 | 0.082 |
| | phenytoin | | | | | |
| NE3A^{NES+BSA2%} | Nestorone | 2.23 | 100 | 0.074 | 80 | 0.092 |

BSA, bovine serum albumin; CHOL, cholesterol; NES, Nestorone (segesterone acetate); FOS, fosphenytoin; PDI, polydispersity index; PHT, phenytoin; RT, room temperature, (close to 20° C.); Z-Ave, mean hydrodynamic diameter (by cumulant analysis).

In one embodiment, the release kinetics of phenytoin in the formula NE3A^{PHT+BSA1%}, evaluated in vitro in Ussing chambers, revealed a slow release (FIG. 2) compared to a solution of the same drug (0.2 mg/g) in 30% ethylene glycol monoethyl ether. The assay was performed at 32° C., with nasal fluid simulating buffer (7 mM sodium phosphate monobasic, 3 mM sodium phosphate dibasic, 30 mM potassium chloride, 107 mM sodium chloride, 1.5 mM calcium chloride, 0.75 mM magnesium chloride, and 5 mM sodium hydrogen carbonate) and 1% albumin (w/w) in the receiving chamber. Characterization of the formulation subjected to the test revealed a slight increase in droplet size to 100 or more nanometers, the PDI also increased but remained between 0.1 and 0.19.

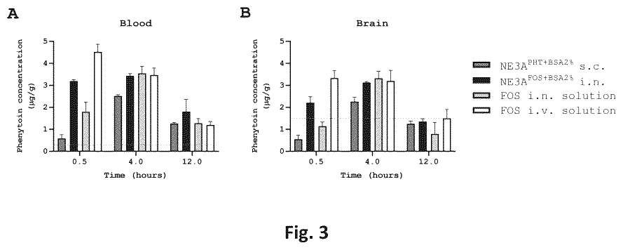

In one embodiment, subcutaneous administration (s.c.) of the formula NE3A^{PHT+BSA2%}, delivering phenytoin as a low aqueous solubility drug model, and i.n. administration of the formula NE3A^{FOS+BSA2%}, delivering fosphenytoin (FOS) as a high aqueous solubility drug model and low permeability, were evaluated in mice at 30 min, 4 h and 12 h post-administration, compared to a solution of fosphenytoin administered by the i.v. and i.n. routes (FIG. 3). Phenytoin nanoemulsion (NE3A^{PHT+BSA2%}) s.c. resulted in lower blood levels than the others at short times (30 min and 4 h), while i.n. administration of fosphenytoin nanoemulsion (NE3A^{FOS+BSA2%}) resulted in levels higher than the other two (NE3A^{PHT+BSA2%} s.c. and FOS i.n.), and originated only lower drug concentrations than FOS i.v. administration at 30 minutes. Brain concentrations were lower than blood concentrations, but followed the same trend.

In an embodiment, formulations comprising cationic lipids were developed. These formulations comprise cetalkonium chloride or dioleoyl-3-trimethylammonium propane (DOTAP) added in a small proportion to the preconcentrate. It was surprisingly found that formulations containing these cationic lipids form nanoemulsions that are already homogeneous or very homogeneous at room temperature, not requiring refrigeration. The composition and characterization of such formulations are depicted in Table 13.

TABLE 13
Composition and droplet size characterization of cationic
nanoemulsions examples (without drugs). Preconcentrate was formed by propylene
glycol monocaprylate:glycerol monooleate: Kolliphor RH 40 in a 3:2:1 mass ratio plus
the cationic lipid at the indicated final mass proportion. As external phase (52%) water or an aqueous
solution of PEG4000 at 4% was used, as indicated.

| | Cationic lipid final | | | | | | |
|---|---|---|---|---|---|---|---|
| | concentration (%, | | | RT | | 4° C. | |
| | w/w) | | Aqueous | Z-Ave | | Z-Ave | |
| Formulation code | Cet.Cl | DOTAP | phase | (nm) | PDI | (nm) | PDI |
| NE3A+^{·Cet.Cl 0.05%} | 0.05 | — | Water | 109.6 | 0.094 | 120 | 0.342 |
| NE3A+^{·Cet.Cl 0.125%} | 0.125 | — | Water | 94.2 | 0.074 | 103.3 | 0.104 |
| NE3A+^{·Cet.Cl 0.25%} | 0.25 | — | Water | 86.2 | 0.084 | 94.3 | 0.237 |
| NE3A+^{·Cet.Cl 0.5%} | 0.5 | — | Water | 126.9 | 0.208 | 154.4 | 0.462 |
| NE3A+^{·Cet.Cl 0.125% PEG 2%} | 0.125 | — | PEG 4% | 126.3 | 0.103 | 116.8 | 0.071 |
| NE3A+^{·Cet.Cl 0.25% PEG 2%} | 0.25 | — | PEG 4% | 133.5 | 0.107 | 104.9 | 0.116 |
| NE3A+^{·Cet.Cl 0.5% PEG 2%} | 0.5 | — | PEG 4% | 157.2 | 0.269 | 133.5 | 0.324 |
| NE3A+^{·DOTAP 0.25%} | — | 0.25 | Water | 96.5 | 0.064 | 118.2 | 0.117 |
| NE3A+^{·DOTAP 0.5%} | — | 0.5 | Water | 142.3 | 0.202 | 163.3 | 0.282 |
| NE3A+^{.DOTAP 1%} | — | 1 | Water | 148 | 0.465 | — | — |

Cet.Cl, cetalkonium chloride; DOTAP, dioleoyl-3-trimethylammonium propane; PDI, polydispersity index; PEG, polyethylene glycol 4000; RT, room temperature, (close to 20° C.); Z-Ave, mean hydrodynamic diameter (by cumulant analysis).

In an embodiment, formulations comprising the cationic lipid cetalkonium chloride were prepared to include the drug simvastatin. It was surprisingly found that formulations containing this cationic lipid remain very homogeneous (PDI<0.1), either at room temperature or upon refrigeration, with the stable incorporation of high concentrations of the drug simvastatin, up to 19.88% (w/w) on the preconcentrate. The composition and characterization of such formulations are depicted in Table 14.

TABLE 14
Composition and droplet size characterization of cationic nanoemulsions examples containing the drug simvastatin.
Preconcentrate was formed by propylene glycol monocaprylate:glycerol monooleate: Kolliphor RH 40:Cetalkonium
chloride in a 2.97:2:1:0.03 mass plus the dissolved drug. The nanoemulsion was prepared
with 50% external phase of malate buffer (30 mM, pH 5), and in one case it was further added PVP for increased viscosity.

| | Final drug | | | | | | 4° C. + 30 | | |
|---|---|---|---|---|---|---|---|---|---|
| | concentration | | RT | | 4° C. | | min RT | | Zeta |
| | (simvastatin) | | Z-Ave | | Z-Ave | | Z-Ave | | potential |
| Formulation code | (w/w) | PVP | (nm) | PDI | (nm) | PDI | (nm) | PDI | (mV) |
| NE3A+^{Cet.Cl 0.5% SIM 5.66%} | 5.66% | 0 | 112.5 | 0.097 | 117.6 | 0.069 | 106.4 | 0.058 | — |
| (sample A)) | | | | | | | | | |
| NE3A+^{Cet.Cl 0.5% SIM 5.66%} | 5.66% | 0 | 128.4 | 0.107 | 108.5 | 0.079 | — | — | 15.1 |
| (sample B) | | | | | | | | | |
| NE3A+^{Cet.Cl 0.5% SIM 7.58%} | 7.58% | 0 | 117.8 | 0.094 | 116.4 | 0.074 | 114.2 | 0.059 | — |
| NE3A+^{Cet.Cl 0.5% SIM 9.94%} | 9.94% | 0 | 116.7 | 0.065 | * | — | — | — | — |
| NE3A+^{Cet.Cl 0.5% SIM 7.76%/PVP} | 7.76% | 0.25% | — | — | 118.3 | 0.079 | — | — | 21.5 |

* Refrigeration is not possible at this drug concentration because it causes the drug to precipitate. RT, room temperature, (close to 20° C.).

The physical stability of the nanoemulsion NE3A+^{Cet.CI 0.5% SIM 5.66%} was demonstrated to be maintained at least for 22 days), at room temperature and at 4° C. (Table 15).

TABLE 15
Composition and droplet size characterization over
time of NE3A+^{Cet. CI 0.5% SIM 5.66%} cationic nanoemulsion.
Preconcentrate was formed by propylene glycol monocaprylate:glycerol
monooleate:Kolliphor RH 40:Cetalkonium chloride in
a 2.97:2:1:0.03 mass plus the dissolved drug for
a final concentration of 5.66% (w/w). The nanoemulsion
was prepared with 50% external phase of malate buffer
(30 mM, pH 5).

| | | |
|---|---|---|
| Time | RT | 4° C. |

| (days) | Z-Ave (nm) | PDI | Z-Ave (nm) | PDI |
|---|---|---|---|---|
| 0 | 106.9 | 0.08 | — | — |
| 1 | 133.5 | 0.108 | — | — |
| 3 | — | — | 125.7 | 0.069 |
| 5 | 144.2 | 0.11 | 126.7 | 0.089 |
| 22 | 115.4 | 0.075 | 115 | 0.059 |

RT, room temperature, (close to 20° C.).

In an embodiment, formulations comprising sodium chloride (NaCl) instead or in addition to a hydrophilic polymer (BSA) were developed. They may comprise the preconcentrate from 50% (w:w) to very low percentages. The formulations were developed to be isotonic with 2.14% (w/w) of preconcentrate and 20 mM phosphate buffer (pH 6-7) plus NaCl at 0,6% (w/w) in the aqueous phase, and may also include for example BSA at different concentrations with significant change in osmolality.

It was surprisingly found that formulations containing sodium chloride (NaCl) in the buffered aqueous phase, even in the absence of any hydrophilic polymer, become already very homogeneous nanoemulsion (PDI<0.1) even at room temperature, and even highly homogeneous after refrigeration (PDI<0.05). The composition and characterization of such formulations are depicted in Table 16.

TABLE 16
Composition and droplet size characterization of variants of formula
NE3A with NaCl in the aqueous phase. Preconcentrate (50% w/w) formed by propylene
glycol monocaprylate: glycerol monooleate: Kolliphor RH 40 in a 3:2:1 mass ratio. The
indicated pH of the external aqueous phase was buffered by 20 nM of sodium hydrogen phosphate.

| | External aqueous phase | | | | | |
|---|---|---|---|---|---|---|
| | composition (50% w/w) | | RT | | 4° C. | |
| | | NaCl | Z-Ave | | Z-Ave | |
| Formulation code | BSA pH 7 | pH 7 | (nm) | PDI | (nm) | PDI |
| NE3A^{BSA 1% NaCl pH7} | 2% | 0.6% | 82.84 | 0.085 | 85.39 | 0.037 |
| | | w/w | | | | |
| NE3A ^{NaCl pH7} | — | 0.6% | 94.67 | 0.085 | 74.38 | 0.037 |
| | | w/w | | | | |

BSA, Bovine serum albumin; PDI, polydispersity index; RT, room temperature, (close to 20° C.); Z-Ave, mean hydrodynamic diameter by cumulant analysis.

In an embodiment, formulations comprising NaCl, with and without BSA at different concentrations and pH values, were prepared diluted (2.14% w/w of preconcentrate) and further comprising the drug nestorone at a final concentration of 0.48 mg/ml These formulations were also very homogeneous (PDI<0.1), even more homogeneous that the equivalent formulations without the drug. The composition and characterization of such formulations are depicted in Table 17.

TABLE 17
Composition and droplet size characterization of variants of diluted
formula NE3A with BSA and/or NaCl in the aqueous phase. Preconcentrate (2.14%)
formed by propylene glycol monocaprylate: glycerol monooleate: Kolliphor RH 40 in a
3:2:1 mass ratio. The indicated pH of the aqueous external phase was buffered by 20 nM of sodium
hydrogen phosphate.

| | | External aqueous phase | | | | |
|---|---|---|---|---|---|---|
| | Nestorone final | composition | | | RT | |
| | concentration | | BSA | NaCl | Z-Ave | |
| Formulation code | (mg/ml) | pH | (w/w) | (w/w) | (nm) | PDI |
| NE3A dil.^{BSA 2% NaCl 0.6% pH6} | 0 | 6 | 2% | 0.6% | 160.9 | 0.126 |
| NE3A dil.^{BSA 0.1% NaCl 0.6% pH6} | 0 | 6 | 0.1% | 0.6% | 142.4 | 0.147 |
| NE3A dil.^{NaCl 0.6% pH6} | 0 | 6 | — | 0.6% | 103.1 | 0.103 |
| NE3A dil.^{BSA 2% NaCl 0.6% pH7} | 0 | 7 | 2% | 0.6% | 142.9 | 0.083 |
| NE3A dil.^{BSA 0.1% NaCl 0.6% pH7} | 0 | 7 | 0.1% | 0.6% | 130.9 | 0.100 |
| NE3A dil.^{NaCl 0.6% pH7} | 0 | 7 | — | 0.6% | 128.9 | 0.078 |
| NE3A dil.^{BSA 2% NaCl 0.6% pH6} | 0.48 | 6 | 2% | 0.6% | 132.4 | 0.075 |
| NE3A dil.^{BSA 0.1% NaCl 0.6% pH6} | 0.48 | 6 | 0.1% | 0.6% | 119.3 | 0.071 |
| NE3A dil.^{NaCl 0.6% pH6} | 0.48 | 6 | — | 0.6% | 124.3 | 0.070 |
| NE3A dil.^{BSA 2% NaCl 0.6% pH7} | 0.48 | 7 | 2% | 0.6% | 133.4 | 0.077 |
| NE3A dil.^{BSA 0.1% NaCl 0.6% pH7} | 0.48 | 7 | 0.1% | 0.6% | 145.8 | 0.111 |
| NE3A dil.^{NaCl 0.6% pH7} | 0.48 | 7 | — | 0.6% | 108.6 | 0.054 |

NE3A dil., NE3A diluted; BSA, Bovine serum albumin; PDI, polydispersity index; RT, room temperature, (close to 20° C.); Z-Ave, mean hydrodynamic diameter by cumulant analysis.

The term “comprising” or “comprises” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof. The above described embodiments are combinable.

Where ranges are provided, the range limits are included. Furthermore, it should be understood that unless otherwise indicated or otherwise evident from the context and/or understanding of a technical expert, the values which are expressed as ranges may assume any specific value within the ranges indicated in different achievements of the invention, at one tenth of the lower limit of the interval, unless the context clearly indicates the contrary. It should also be understood that, unless otherwise indicated or otherwise evident from the context and/or understanding of a technical expert, values expressed as range may assume any sub-range within the given range, where the limits of the sub-range are expressed with the same degree of precision as the tenth of the unit of the lower limit of the range.

The following dependent claims further set out particular embodiments of the disclosure.

### REFERENCES

1. Corsini A, Bellosta S, Baetta R, Fumagalli R, Paoletti R, Bernini F. New insights into the pharmacodynamic and pharmacokinetic properties of statins. Pharmacol Ther. December 1999; 84(3):413-28.
2. Armitage J. The safety of statins in clinical practice. The Lancet. November 2007; 370(9601):1781-90.
3. MRC/BHF Heart Protection Study Collaborative Group. Effects of simvastatin 40 mg daily on muscle and liver adverse effects in a 5-year randomized placebo-controlled trial in 20,536 high-risk people. BMC Clin Pharmacol. December 2009; 9(1):6.
4. Galtier F, Mura T, Raynaud de Mauverger E, Chevassus H, Farret A, Gagnol J P, et al. Effect of a high dose of simvastatin on muscle mitochondrial metabolism and calcium signaling in healthy volunteers. Toxicol Appl Pharmacol. September 2012; 263(3):281-6.
5. Crowe T P, Greenlee M H W, Kanthasamy A G, Hsu W H. Mechanism of intranasal drug delivery directly to the brain. Life Sci. Feb. 15, 2018; 195:44-52.
6. Chavhan S S, Petkar K C, Sawant K K. Simvastatin nanoemulsion for improved oral delivery: Design, characterization, in vitro and in vivo studies. J Microencapsul. 2013; 30(8):771-9.
7. Nazir A, Schroën K, Boom R. Premix emulsification: A review. J Member Sci. 2010; 362(1-2):1-11.
8. Pires P C, Santos L T, Rodrigues M, Alves G, Santos A O. Intranasal phosphenytoin: The promise of phosphate esters in nose-to-brain delivery of poorly soluble drugs. Int J Pharm. November 2021; 592:120040.
9. Sonvico F, Garrastazu G, Batger M, Rondelli V, Cantù L, Del Favero E, et al. The nasal delivery of nanoencapsulated statins &ndash; an approach for brain delivery. Int J Nanomedicine. 2016; 11:6575-90.
10. Bruinsmann F A, Pigana S, Aguirre T, Souto G D, Pereira G G, Bianchera A, et al. Chitosan-coated nanoparticles: Effect of chitosan molecular weight on nasal transmucosal delivery. Pharmaceutics. 2019; 11(2).
11. Manickavasagam D, Novak K, Oyewumi M O. Therapeutic Delivery of Simvastatin Loaded in PLA-PEG Polymers Resulted in Amplification of Anti-inflammatory Effects in Activated Microglia. AAPS J. 2018; 20(1):1-13

1. A self-emulsifying composition comprising
9-19% (w/w) of a neutral hydrophilic surfactant and;
81-91% (w/w) of a combination of at least two hydrophobic excipients in a mass ratio between 4:1 and 1.2:1,
propylene glycol monocaprylate as the first excipient;
a second excipient selected from the following list: glycerol monooleate, miglyol 812; glycerol monocaprylocaprate, soybean oil, type I glycerol monocaprylate, sorbitan monooleate, decyl oleate, glycerol monolinoleate, vitamin E, or mixtures thereof.

2. The composition according to the claim 1 further comprising an active agent, wherein the active agent is a pharmaceutical active agent or a cosmetic active agent; wherein the active agent selected from a list consisting of: a hydrophilic agent, and whose permeation of biological barriers such as mucous membranes and consequently absorption and bioavailability are improved by the formulation, a statin, a steroid compound and/or an antiepileptic.

3.-5. (canceled)

6. The composition according to claim 2 wherein the active agent is selected from a list comprising: atorvastatin, rosuvastatin, pravastatin, lovastatin, fluvastatin, pitavastatin, simvastatin, phenytoin, fosphenytoin, progesterone, nestorone or mixtures thereof.

7. The composition according to claim 1 wherein the neutral hydrophilic surfactant is macrogolglycerol hydroxystearate.

8. The composition according to claim 1 further comprising a cationic lipid; preferably wherein the cationic lipid is selected from a list consisting of cetalkonium chloride and dioleoyl-3-trimethylammonium propane.

9. The composition according to claim 1 wherein the concentration of the cationic lipid ranges from 0.01% to 1% (w/w); preferably 0.05 to 0.5% (w/w).

10. An aqueous emulsion comprising the self-emulsifying composition according claim 1 wherein the concentration of the aqueous self-emulsifying composition is at least 0.1% (w/w) and at least 50% (w/w) of water, wherein 90% of the droplets comprise a dimension less than 200 nm, wherein the polydispersity index after cooling at 2-8° C. is less than or equal to 0.2.

11.-13. (canceled)

14. The aqueous emulsion according to claim 10, wherein 50% of the droplets comprise a dimension ranging from 90 to 120 nm.

15. (canceled)

16. The aqueous emulsion according to claim 10 wherein the polydispersity index after cooling at 2-8° C. is less than or equal to 0.1.

17. The aqueous emulsion according to claim 10 further comprising a hydrophilic polymer.

18. (canceled)

19. The aqueous emulsion according to claim 17 wherein the hydrophilic polymer is selected from a list consisting of: albumin, polyvinylpyrrolidone, hydroxypropylmethylcellulose, polyethylene glycol, or mixtures thereof.

20. The aqueous emulsion according to claim 10 further comprising a salt; preferably the salt is sodium chloride.

21. (canceled)

22. The aqueous emulsion according to claim 10 wherein the concentration of the active agent ranges up to 100 mg/g.

23.-25. (canceled)

26. The aqueous emulsion according to claim 10, wherein the emulsion remains homogeneous between 2 and 37° C.

27. The aqueous emulsion according to claim 10 wherein the conditions being guaranteed that promote chemical and microbiological stability of the preparation; the emulsion maintains physical stability when stored at 2 to 25° C. for long periods of time; preferably periods longer than 6 months; more preferably for periods longer than 1 year.

28. A pharmaceutical composition comprising the self-emulsifying composition according to claim 1.

29. (canceled)

30. The pharmaceutical composition according to claim 28 for enteral, parenteral or intranasal administration, preferably for intranasal administration.

31. A cosmetic composition comprising the self-emulsifying composition according to claim 1.

32. (canceled)
